(19) United States
(12) Patent Application Publication
(10) Pub. No.: US 2004/0197838 A1
(43) Pub. Date: Oct. 7, 2004

(54) Phosphorylated histone h2b as apoptosis marker

(76) Inventors: C David Allis, New York, NY (US); Wang L Cheung, Charlottesville, VA (US); Ajiro Kozo, Chiba-ken (JP)

Correspondence Address: UNIVERSITY OF VIRGINIA PATENT FOUNDATION, 1224 WEST MAIN STREET, SUITE 1-110, CHARLOTTESVILLE, VA, 22903, US

(21) Appl. No.: 10/485,422

(22) PCT Filed: Aug. 1, 2002
(86) PCT No.: PCT/US02/24405

Related U.S. Application Data
(60) Provisional application No. 60/310,015, filed on Aug. 3, 2001.
(60) Provisional application No. 60/364,386, filed on Mar. 14, 2002.

(51) Int. Cl. G01N 33/574; C07K 7/08; C07K 16/46

(52) U.S. Cl. 435/7.23; 530/326; 530/327; 530/328; 530/391.1

(57) ABSTRACT

The present invention is directed to the generation of antibodies against an α-phosphorylated serine 14 residue on Histone 2B. This postranslational modification of the amino terminus of Histone 2B is associated with cells that are about to enter or are already undergoing apoptosis. These antibodies are useful in identifying apoptotic cells and serve as diagnostic and screening tools. The present invention is also directed to the Mst1 kinase and the isolation of compounds that modulate the activity of Mst1. This kinase has been identified as the enzyme responsible for creating this postranslational modification on Histone 2B in vivo.

### RELATED APPLICATION

This application claims priority under 35 USC §199(e) to U.S. Provisional Application Serial No. 60/310,015, filed Aug. 3, 2001, 60/364,386, filed Mar. 14, 2002 and 60/375,892, filed Apr. 26, 2002, the disclosures of which are incorporated herein.

### US GOVERNMENT RIGHTS

This invention was made with United States Government support under Grant No. GM 40922, awarded by the National Institutes of Health. The United States Government has certain rights in the invention.

### FIELD OF THE INVENTION

The present invention is directed to antibodies that bind to histone epitopes created by postranslational modification of the histone protein, compositions comprising such antibodies, and the use of such compositions as diagnostic and screening tools. More particularly, the present invention is directed to a histone modification that is associated with apoptosis.

### BACKGROUND OF THE INVENTION

Apoptosis is programmed cell death, a naturally occurring process involved in both the development and aging of cells. It is the process whereby the body can rid itself of unwanted, old, or damaged cells. Apoptosis is the physiological counterpart of cell proliferation. It is essential for both biological processes such as normal tissue turnover, embryonic development, and maturation of the immune system, including pathological processes, such as hormone deprivation, thermal stress and metabolic stress. (Wyllie, A. H., in Bowen and Lockshin (eds.) Cell Death in Biology and Pathology (Chapman and Hall, 1981), at 9-34).

As more is learned of programed cell death it is becoming apparent that there may be several different mechanisms in which programmed cell death occurs. In one embodiment, the cell death is characterized by a decrease in cell volume, a condensation of chromatin, cellular budding, and the fragmentation of DNA into a ladder of 180 base pair (bp) oligomers with 3′-OH free ends, a hallmark of apoptosis. Cell membranes maintain their integrity through the process, and lysosomes remain intact. No inflammatory response occurs during the process of apoptosis. Affected cells undergo phagocytosis by adjacent normal cells and by some macrophages. Alternatively, other mechanisms of cell death have recently been described wherein the cell organelles themselves are fragmented. As used herein the term “apoptosis” is intended to cover all forms of programed cell death.

The biochemical effector pathways that underlie the apoptotic mechanisms are as yet unknown. It has been suggested that the apoptotic mechanism involves one or more Ca²⁺/Mg²⁺-dependent endogenous endonucleases (Arends et al., (1990) Am. J. Pathol. 136:593-608); transglutaminase activity (Fesus et al., (1987) FEBS Lett. 224:104-108; Taress et al., (1992) J. Biol. Chem. Cell 75:653-660); and the generation of oxygen radicals (Hockenberry et al., (1993) Cell 75:241-251; Butke and Sandstrom (1994) Immun. Today 15:7-10). It appears that gene expression is required for apoptosis as this process can be stopped by inhibitors of RNA or protein synthesis (Martin et al., (1988) J. Cell Biol. 106:829-844).

Apoptosis can be activated by a number of intrinsic or extrinsic signals. These signals include the following: mild physical signals, such as ionization radiation, ultraviolet radiation, or hyperthermia; low to medium doses of toxic compounds, such as azides or hydrogen peroxides; chemotherapeutic drugs, such as etoposides and teniposides, cytokines such as tumour necrosis factors and transforming growth factors; and stimulation of T-cell receptors.

The fundamental unit of eukaryotic chromatin is the nucleosome, a particle containing 146 base pairs of DNA wrapped around a histone core octamer (two copies each of histone H3, H4, H2A, and H2B). How nucleosomal arrays are then packaged into higher-order chromatin fibers that, in turn, define distinct functional domains is poorly understood. An increasing body of evidence suggests that histone post-translational modifications (acetylation, phosphorylation, methylation, etc.) provide one mechanism that is central to this process in some instances. In addition, recent evidence suggest that distinct patterns of covalently-modified histones can act as ‘signaling platforms’ (i.e. a ‘histone code’) to recruit and bind nuclear factors that mediate downstream functions by mechanisms that remain unclear.

Given the intimate association between histones and DNA, changes in the integrity of DNA and the state of chromatin compaction that characterize apoptosis may be mediated by histone modifications. However, to date, histone modifications that are specifically induced during apoptosis remain poorly defined. Mitotic chromatin condensation, for example, is associated with histone H3 phosphorylation at serine 10, but this modification has not been consistently observed during apoptotic-induced chromatin condensation (reviewed in Cheung et al., (2000) Cell 103, 263-271. In contrast, phosphorylation of a relatively minor histone variant, H2A.X, increases during early stages of DNA fragmentation in apoptosis (Rogakou et al., (2000) J Biol Chem 275, 9390-9395). However, H2A.X phosphorylation (at serine 139) correlates with all known double-stranded DNA breaks suggesting that it acts more as a ‘DNA-damage sensor’ than a specific chromatin mark linked to the apoptotic process.

In mammalian cells, the only major core histone modification that has been uniquely associated with apoptosis is histone H2B phosphorylation, although the responsible sites of phosphorylation in its amino terminus are poorly defined. In keeping, the H2B amino terminal tail, but not other bistone tails, is essential for chromatin condensation in Xenopus cell-free systems that induce chromatin condensation.

The present invention is directed to a specific histone (H2B) post-translational modification (serine14 phosphorylation) that specifically correlates with the onset of apoptotic chromatin condensation and DNA fragmentation in human HL-60 cells. Furthermore, the present invention describes an antibody that specifically binds to this apoptosis marker (hereafter the “S14P antibody”) and the use of that antibody to diagnose and treat various disease states.

When apoptosis is unregulated, disease results. Unregulated apoptosis is involved in diseases such as cancer, heart disease, neurodegenerative disorders, autoimmune disorders, and viral and bacterial infections. Cancer, for example, not only triggers cells to proliferate but also blocks apoptosis. Cancer is partly a failure of apoptosis wherein the orders for the cells to kill themselves by apoptosis are blocked. New cancer treatments that involve inducing apoptosis are being researched.

### SUMMARY OF THE INVENTION

The present invention is directed to a marker uniquely associated with programmed cell death. The marker is a post translational modification (phosphorylation of serine 14) of the amino-terminus of histone H2B. More particularly, the present invention is directed to antibodies that specifically bind to the epitope created by the modification of histone H2B and serve as markers of cells undergoing apoptosis. The present invention is also directed to the kinase, Mst1, responsible for phosphorylating serine 14 of histone H2B and assays for identifying compounds capable of modifying Mst1 activity.

### BRIEF DESCRIPTION OF THE DRAWINGS

FIG. 1 shows the data from an ELISA demonstrating that the S14P antibody recognizes only the H2B phosphoserine 14 peptides, and this activity can be inhibited by the H2B phosphoserine 14 peptide but not the unmodified peptide. The peptides were coated onto ELISA plates in the indicated amounts by incubating the peptides in PBS overnight at 4° C. The well was blocked using PBST (PBS containing 0.5% Tween-20) for 1 hour and washed three times with PBST. Anti-phos(S14) H2B serum was added at 1:2000 dilution to each well for 2 hours and then followed by more washing. Horseradish peroxidase conjugated rabbit secondary antibody at 1:5000 in PBST was added to each well for 2 hours. After three washes using PBST, o-phenylenediamine dihydrochloride peroxidase substrate (Sigma) was added and quantitated by absorbance at OD of 492 nm. The peptides immobilized and tested include: S14 (SEQ ID NO: 2; □); S14P (SEQ ID NO: 3; ⋄); S14P with ELISA conducted using sera pre-incubated with 1 mg/ml of S14 competitor peptide prior to the ELISA. (◯); S14P with ELISA conducted using sera pre-incubated with 1 mg/ml of S14P competitor peptide prior to the ELISA (Δ).

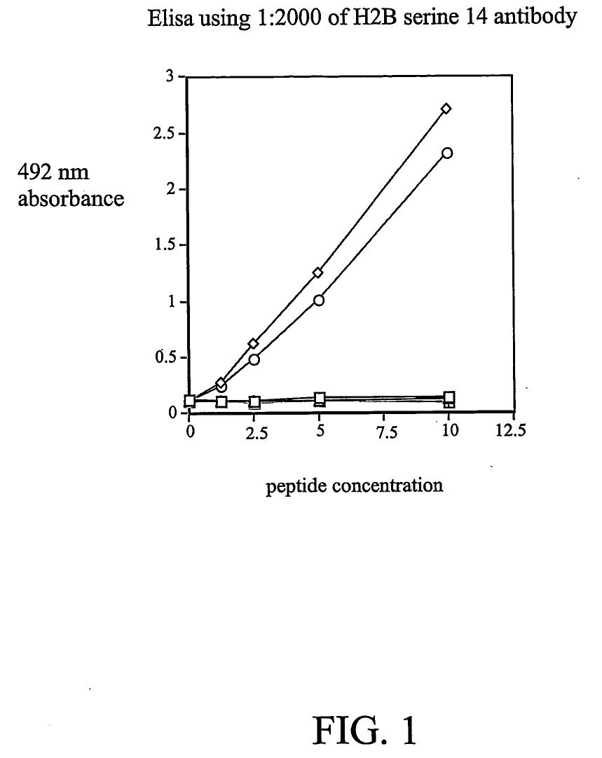

### DETAILED DESCRIPTION OF THE INVENTION

Definintions

In describing and claiming the invention, the following terminology will be used in accordance with the definitions set forth below.

As used herein, the term “nucleic acid” encompasses RNA as well as single and double-stranded DNA and cDNA. Furthermore, the terms, “nucleic acid,” “DNA,” “RNA” and similar terms also include nucleic acid analogs, i.e. analogs having other than a phosphodiester backbone. For example, the so-called “peptide nucleic acids,” which are known in the art and have peptide bonds instead of phosphodiester bonds in the backbone, are considered within the scope of the present invention.

The term “peptide” encompasses a sequence of 3 or more amino acids wherein the amino acids are naturally occurring or synthetic (non-naturally occurring) amino acids. Peptide mimetics include peptides having one or more of the following modifications:

1. peptides wherein one or more of the peptidyl—C(O)NR—linkages (bonds) have been replaced by a non-peptidyl linkage such as a—CH₂-carbamate linkage (—CH₂OC(O)NR—), a phosphonate linkage, a —CH₂-sulfonamide (—CH 2-S(O)₂NR—) linkage, a urea (—NHC(O)NH—) linkage, a—CH₂-secondary amine linkage, or with an alkylated peptidyl linkage (—C(O)NR—) wherein R is C₁-C₄ alkyl;

2. peptides wherein the N-terminus is derivatized to a—NRR₁ group, to a—NRC(O)R group, to a—NRC(O)OR group, to a—NRS(O)₂R group, to a—NHC(O)NHR group where R and R₁ are hydrogen or C₁-C₄ alkyl with the proviso that R and R₁ are not both hydrogen;

3. peptides wherein the C terminus is derivatized to —C(O)R₂ where R₂ is selected from the group consisting of C₁-C₄ alkoxy, and —NR₃R₄ where R₃ and R₄ are independently selected from the group consisting of hydrogen and C₁ C₄ alkyl.

Naturally occurring amino acid residues in peptides are abbreviated as recommended by the IUPAC-IUB Biochemical Nomenclature Commission as follows: Phenylalanine is Phe or F; Leucine is Leu or L; Isoleucine is Ile or I; Methionine is Met or M; Norleucine is Nle; Valine is Vat or V; Serine is Ser or S; Proline is Pro or P; Threonine is Thr or T; Alanine is Ala or A; Tyrosine is Tyr or Y; Histidine is His or H; Glutamine is Gln or Q; Asparagine is Asn or N; Lysine is Lys or K; Aspartic Acid is Asp or D; Glutamic Acid is Glu or E; Cysteine is Cys or C; Tryptophan is Trp or W; Arginine is Arg or R; Glycine is Gly or G, and X is any amino acid. Other naturally occurring amino acids include, by way of example, 4-hydroxyproline, 5-hydroxylysine, and the like.

As used herein, the term “conservative amino acid substitution” is defined herein as exchanges within one of the following five groups:

I. Small aliphatic, nonpolar or slightly polar residues:

Ala, Ser, Thr, Pro, Gly;

II. Polar, negatively charged residues and their amides:

Asp, Asn, Glu, Gln;

III. Polar, positively charged residues:

His, Arg, Lys;

IV. Large, aliphatic, nonpolar residues:

Met Leu, ile, Val, Cys

V. Large, aromatic residues:

Phe, Tyr, Trp

As used herein, the term “purified” and like terms relate to the isolation of a molecule or compound in a form that is substantially free (i.e. at least 60% free, preferably 80% free, and most preferably greater than 90% free) from other components with which they are naturally associated.

“Operably linked” refers to a juxtaposition wherein the components are configured so as to perform their usual function. For example, control sequences or promoters operably linked to a coding sequence are capable of effecting the expression of the coding sequence.

As used herein the term “solid support” relates to a solvent insoluble substrate that is capable of forming linkages (preferably covalent bonds) with soluble molecules. The support can be either biological in nature, such as, without limitation, a cell or bacteriophage particle, or synthetic, such as, without limitation, an acrylamide derivative, glass, plastic, agarose, cellulose, nylon, silica, or magnetized particles. The support can be in particulate form or a monolythic strip or sheet. The surface of such supports may be solid or porous and of any convenient shape.

As used herein, the terms “complementary” or “complementarity” are used in reference to polynucleotides (i.e., a sequence of nucleotides) related by the base-pairing rules. For example, for the sequence “A-G-T,” is complementary to the sequence “T-C-A.”

“Therapeutic agent,” “pharmaceutical agent” or “drug” refers to any therapeutic or prophylactic agent which may be used in the treatment (including the prevention, diagnosis, alleviation, or cure) of a malady, affliction, disease or injury in a patient.

As used herein, the term “treating” includes alleviating the symptoms associated with a specific disorder or condition and/or preventing or eliminating said symptoms. For example, treating cancer includes preventing or slowing the growth and/or division of cancer cells as well as killing cancer cells.

As used herein, the term “pharmaceutically acceptable carrier” encompasses any of the standard pharmaceutical carriers, such as a phosphate buffered saline solution, water and emulsions such as an oil/water or water/oil emulsion, and various types of wetting agents.

As used herein, the term “antigenic fragment of H2B serine 14” encompasses both natural peptide fragments of the amino terminus of histone H2B (including the peptide fragments SAPAPKKGSKK (SEQ ID NO: 1) and SAPAPKKGSKK (SEQ ID NO: 2)) and synthetic equivalents of those fragments.

As used herein, the term “antibody” refers to a polyclonal or monoclonal antibody or a binding fragment thereof such as Fab, F(ab′)₂ and Fv fragments.

As used herein the letter S in bold face type (S), when used in the context of an amino acid sequence, will represent a serine amino acid that has been phosphorylated. The terms “Ser(P)” of “S(P)” as used herein, also refer to the phosphorylated form of the amino acid serine.

As used herein, the term “S14P antibody” is an antibody that binds specifically to the sequence CSAPAPKKGSKK (SEQ ID NO: 3); and the term “S14 antibody” is an antibody that binds specifically to the sequence CSAPAPKKGSKK (SEQ ID NO: 2).

As used herein, the term “biologically active fragments” of the S14P antibody encompasses natural or synthetic portions of the respective full-length antibody that are capable of specific binding to the peptide CSAPAPKKGSKK (SEQ ID NO: 3).

A “linker” is a molecule (or group of molecules) that serves to chemically link two disparate entities. For example a peptide linker chemically links two polypeptides via a peptide bond.

As used herein, the term “parenteral” includes administration subcutaneously, intravenously or intramuscularly.

The Invention

The present invention is directed to the discovery that modification of the amino terminus of the histone H2B protein, and homologous proteins from other species, can serve as a marker of apoptosis. More particularly, applicants have discovered that the amino terminus of histone H2B protrudes from the surface of the chromatin and the serine amino acid at the 14th position from the amino terminus (Ser14) is selectively phosphorylated in vivo in cells that will undergo or have already begun the process of apoptosis. Therefore, in accordance with one aspect of the present invention phosphorylation of Ser14 of histone H2B serves as a marker of apoptosis in vertebrate species, and antibodies recognizing this portion of the protein have use as important diagnostic tools.

One aspect of the present invention is directed to antigens used to produce antibodies specific to the amino terminus of the phosphorylated histone H2B protein (Phos H2B). In one embodiment, a purified antigenic fragment of the amino terminus of Phos H2B or a synthetic equivalent thereof is provided. The antigen comprises an amino acid fragment comprising the sequence CSAPAPKKGSKK (SEQ ID NO: 3), wherein C is an artificial cysteine residue added to the native H2B sequence. In one preferred embodiment the antigen consists of the sequence SAPAPKKGSKK (SEQ ID NO: 1) or an amino acid sequences that differ from SAPAPKKGSKK (SEQ ID NO: 1) by one or more conservative amino acid substitutions. The present invention also encompasses an antigen that comprises the non-phosporylated H2B sequence: SAPAPKKGSKK (SEQ ID NO: 2). In an alternative embodiment, the purified antigen comprises a polypeptide linked to a suitable carrier, such as bovine serum albumin or Keyhole limpet hemocyanin. The present invention is also directed to nucleic acid sequences that encode for the peptide sequence SAPAPKKGSKK (SEQ ID NO: 2).

In one preferred embodiment a Phos H2B antibody is provided that binds specifically to the sequence CSAPAPKKGSKK (SEQ ID NO: 3; the “S14P antibody”), and an Unmodified H2B antibody is provided that binds specifically to the sequence CSAPAPKKGSKK (SEQ ID NO: 2; the “S14 antibody”).

One method used to generate the S14P antibody or the S14 antibody involves administration of the respective antigen to a laboratory animal, typically a rabbit, to trigger production of antibodies specific for the antigen. The dose and regiment of antigen administration to trigger antibody production as well as the methods for purification of the antibody are well known to those skilled in the art. Typically, such antibodies can be raised by administering the antigen of interest subcutaneously to New Zealand white rabbits which have first been bled to obtain pre-immune serum. The antigens can be injected at a total volume of 100 ul per site at six different sites. Each injected material will contain synthetic surfactant adjuvant pluronic polyols, or pulverized acrylamide gel containing the protein or polypeptide after SDS-polyacrylamide gel electrophoresis.

The rabbits are then bled two weeks after the first injection and periodically boosted with the same antigen three times every six weeks. A sample of serum is then collected 10 days after each boost. Polyclonal antibodies are then recovered from the serum by affinity chromatography using the corresponding antigen to capture the antibody. Ultimately, the rabbits are euthenized with pentobarbital 150 mg/Kg IV. This and other procedures for raising polyclonal antibodies are disclosed in E. Harlow, et. al., editors, Antibodies: A Laboratory Manual (1988), which is hereby incorporated by reference. The specificity of antibodies may be determined by enzyme-linked immunosorbent assay or immunoblotting, or similar methods known to those skilled in the art.

The present invention also encompasses monoclonal antibodies that specifically bind to either the H2B S14P antigen (i.e. a fragment of the amino terminus of histone H2B containing the phosphorylated serine 14 amino acid) or the unmodified H2B S14 antigen (i.e. a fragment of the amino terminus of histone H2B containing the unphosphorylated serine 14 amino acid). Monoclonal antibody production may be effected using techniques well-known to those skilled in the art. Basically, the process involves first obtaining immune cells (lymphocytes) from the spleen of a mammal (e.g., mouse) which has been previously immunized with the antigen of interest either in vivo or in vitro. The antibody-secreting lymphocytes are then fused with myeloma cells or transformed cells, which are capable of replicating indefinitely in cell culture, thereby producing an immortal, immunoglobulin-secreting cell line. The resulting fused cells, or hybridomas, are cultured, and the resulting colonies screened for the production of the desired monoclonal antibodies. Colonies producing such antibodies are cloned, and grown either in vivo or in vitro to produce large quantities of antibody. One embodiment of the invention is directed to a hybridoma cell line which produces monoclonal antibodies which bind the H2B S14P or the H2B S14 antigens. A description of the theoretical basis and practical methodology of fusing such cells is set forth in Kohler and Milstein, Nature, 256:495 (1975), which is hereby incorporated by reference.

In addition to whole antibodies, fragments of antibodies can retain binding specificity for a particular antigen. Antibody fragments can be generated by several methods, including, but not limited to proteolysis or synthesis using recombinant DNA technology. An example of such an embodiment is selective proteolysis of the H2B S14P or the H2B S14 antibody by papain to generate Fab fragments, or by pepsin to generate a F(ab′)₂ fragment. These antibody fragments can be made by conventional procedures, as described in J. Goding, Monoclonal Antibodies: Principles and Practice, pp. 98-118 (N.Y. Academic Press 1983), which is hereby incorporated by reference. Other fragments of the H2B S14P or the H2B S14 antibodies which retain the specific binding of the whole antibody can be generated by other means known to those skilled in the art.

In one embodiment the antibodies are labeled. It is not intended that the present invention be limited to any particular detection system or label. The antibody may be labeled with a fluorophore, a radioisotope, or a non-isotopic labeling reagent such as biotin or digoxigenin; antibodies containing biotin may be detected using “detection reagents” such as avidin conjugated to any desirable label such as a fluorochrome. In one embodiment the histone specific antibodies of the present invention are indirectly labeled through the use of a secondary antibody, wherein the secondary antibody is labeled and is specific for the primary (histone specific) antibody. Alternatively, the histone specific antibody may be directly labeled with a radioisotope or fluorochrome such as FITC or rhodamine; in such cases secondary detection reagents may not be required for the detection of the labeled probe. The presence of the modified histones in the blood can then be detected through the use of the relevant labeled antibody.

The antibodies or fragments of the present invention can be combined with a carrier or diluent to form a composition. In one embodiment, the carrier is a pharmaceutically acceptable carrier. The term “carrier” refers to a diluent, adjuvant, excipient or vehicle with which an active agent is administered. Such carriers and diluents include sterile liquids such as phosphate buffered saline solution, water, oils and emulsions such as an oil/water or water/oil emulsion, and various types of wetting agents including those agents approved by a regulatory agency of the US Federal government or listed in the US Pharmacopeia for use in animals, including humans. The compositions may further include the addition of a surfactant and other pharmaceutically and physiologically acceptable carrier, including adjuvants, excipients or stabilizers. Illustrative oils are those of petroleum, animal, vegetable, or synthetic origin, for example, peanut oil, soybean oil, or mineral oil. In general, water, saline, aqueous dextrose, and related sugar solution, and glycols such as, propylene glycol or polyethylene glycol, are preferred liquid carriers, particularly for injectable solutions.

Immunoflourescence of HL-60 cells treated with VP16 and stained using the S14P antibody (directed against the peptide CSAPAPKKGSKK) revealed a staining pattern showing that H2B S14 phosphorylation is localized to apoptotic cells as indicated by fragmented DAPI stained nuclei. Apoptosis was induced using etoposide and following the protocol in Kozo Ajiro's paper (Ajiro K. Histone H2B phosphorylation in mammalian apoptotic cells: An association with DNA fragmentation, J Biol. Chem. 2000 Jan. 7;275(1):439-43).

Interestingly, the function of H2B Ser14 phosphorylation with respect to apoptosis appears to apply only to vertebrate species since Ser14 and surrounding sequences are well conserved from frogs to humans, but are not observed in flies or worms. This chromatin modification has been demonstrated in human, mouse and Xenopus cells but staining with α-Phos (Ser 14) H2B (the S14P antibody) was not seen in invertebrates such as C. elegans. Interestingly, Ser32 in H2B also appears to be a ‘vertebrate addition’, and some evidence has suggested that this may also be a site of apoptotic H2B phosphorylation, at least in HL-60 cells. Noteworthy also is Ser36 in the H2B amino terminus, a site which is conserved from yeast to humans and is present in flies and worms. Whether Ser36 in H2B, or the equivalent residue in invertebrates, is phosphorylated during their apoptotic programs is not known. However, the fact that yeast appear to contain this site make it likely that phosphorylation here, if it occurs at all, has other biological functions outside of apoptosis.

Based on time course studies, histone H2B serine 14 phosphorylation occurs immediately before DNA laddering suggesting that histone H2B phosphorylation might be involved in or act as a ‘trigger ’ for DNA fragmentation during apoptosis. Several nucleases that cleave internucleosomal DNA during apoptosis have been identified. At least in vitro, histone H1 and HMG enhance nuclease function, and thus, phosphorylated H2B could lead to chromatin structural alterations that enhance accessibility of key DNase activities to chromatin or facilitate their recruitment to the chromatin.

The compositions comprising the S14P antibody, or a bioactive fragment thereof, and a carrier or diluent can be used in conjunction with a method to detect cells that are destine to enter into apoptosis or cells that already are in apoptosis. In addition, the antibodies of the present invention can be used to detect cells that fail to be induced into the apoptosis process and thus serve as a marker for identifying precancer/cancerous cells and tissues. In accordance with this embodiment, cells are cultured in the presence of an apoptosis inducing agent (such as the etoposide, VP-16) and the cells then fixed and stained to determine if a substantial population of cells fail to enter apoptosis relative to a control population of cells. Such a method can be used to analyze cells recovered in a biopsy to diagnose malignancies and determine the aggressiveness of a tumor and thus the selection of treatment strategies.

In accordance with one embodiment, a method for detecting the presence of apoptotic cells or cells about to become apoptotic in a vertebrate species, including humans is provided. The method comprises the steps of first isolating a biological sample from the vertebrate species, wherein the biological sample comprises the species' chromatin. Typically, the biological sample comprises tissues or cells isolated from the individual vertebrate species. In one embodiment the chromatin (i.e. genomic DNA associated with histones) or individual histones are purified from the biological sample prior to contacting the purified chromatin/histones with the S14P antibody. Alternatively, cells isolated from the vertebrate species can be fixed and directly stained with the antibody.

To determine the presence of apoptotic cells, chromatin or histones isolated from the individual are contacted with the S14P antibody under conditions suitable for specific binding of the antibody to its target. Non-specific bound S14P is subsequently removed and the immunocomplexes formed between the chromatin and the S14P antibody are identified and quantitated as an indicator of the presence of apoptotic cells in the vertebrate species. In one embodiment the S14P antibody is labeled, or alternatively a labeled secondary antibody (specific for the S14P antibody or histone H2B) is used, to identify and quantitate the formation of the immunocomplexes. The amount of immunocomplexes formed is directly proportional to the amount of phosphorylated S14 histone H2B that is present in the sample and is thus indicative of apoptotic activity in the individual that served as a source of the biological sample. The detection of an abnormal level of apoptotic activity serves as a diagnostic of various disease states, including cancer.

In accordance with one embodiment, the method of identifying/quantitating the formation of the immunocomplexes comprises the steps of fixing or binding cells, or the purified chromatin, to a solid support and probing with labeled S14P antibody, wherein non-specifically bound antibody is removed by washing with buffered solutions. Alternatively, the formation of the immunocomplexes can be identified by immunoprecipitation. In another embodiment, the S14P antibody is immobilized on a solid support and the immobilized antibody is contacted with the isolated chromatin/histones. The formation of immunocomplexes is then identified by washing the bound antibody with a buffered solution and contacting the bound antibody with a second labeled antibody that specifically binds to histone H2B at a site separate and distinct from the S14P epitope.

The Mst1(mammalian Ste20-like kinase 1) kinase is a ubiquitously expressed serine/threonine kinase whose cellular function is unknown. Mst1 is a member of the sterile 20-like superfamily of which ˜30 related kinases exist in humans. This kinase is typically regarded as an upstream regulator of MAPK pathways with roles in apoptosis, morphogenesis and cytoskeletal rearrangements. The kinase is know to be cleaved by activated effector caspase 3, releasing a C-terminal regulatory region, and the activated N-terminal catalytic domain. This activated domain then induces apoptosis in a pathway where physiological substrates were previously unknown. Applicants have now discovered that one substrate for Mst1 is the 14th serine residue of histone 2B.

Based on the data in the present application, it is believed that the caspase-cleaved form of Mst1 phosphorylates histone H2B as one of its physiological targets during apoptosis. Covalent modification of the histone termini then facilitates apoptotic chromatin condensation and DNA fragmentation leading to cell death by mechanisms that remain poorly understood. For example, how histone H2B phosphorylation at Ser14 can affect the chromatin structure during apoptosis is not known, although previous work has shown that the histone H2B amino terminus tail is essential for in vitro chromatin condensation. Thus, it is anticipated that H2B phosphorylation is involved in chromatin condensation during apoptosis.

Understanding the mechanism of chromatin changes during apoptosis will provide another avenue for drug targeting that may either induce or inhibit cellular death. Chromatin changes such as condensation and DNA fragmentation have been viewed as the last committed step of apoptosis. Considerable evidence exists suggesting many cells die under stress by undergoing apoptosis. However, the use of caspase inhibitors has not been effective to decrease cell death after the initial stress (such as ischemia) has occurred. Perhaps, after effector caspases initiate the death pathway leading to defined chromatin changes, caspases are no longer needed. Accordingly, effective prevention of cell death may be best brought about by combining caspase inhibitors with drugs that target downstream activities such as Mst1 that affect chromatin changes during apoptosis. In accordance with one embodiment a method is provided for inhibiting cell death by treating cells with caspases inhibitors and an inhibitor of Mst1.

In one embodiment, a method is provided for detecting the kinase activity of a sample, and more particularly Mst1 activity. The method comprises contacting a peptide comprising the sequence CSAPAPKKGSKK (SEQ ID NO: 2) for a predetermined length of time with a sample that is suspected of having kinase activity. The amount of S14P antibody that binds to the substrate is a direct correlation of the extent the substrate was phosphorylated during the predetermined time length and thus indicates the kinase activity of the sample. This assay can also be used to screen for potential modulators of Mst1 activity, including inhibitors and stimulants of Mst1 activity. For example, in one embodiment a method of screening for inhibitors of Mst1 activity comprises the steps of providing a sample, wherein the sample comprises a kinase (i.e. Mst1) and a substrate that is methylated by that kinase (for example the peptide CSAPAPKKGSKK; SEQ ID NO: 2 will be used as the substrate for Mst1 activity), adding a potential inhibitor of the kinase to the sample, and incubating the sample for a predeterminied length of time under conditions that are typically (i.e. in the absence of an inhibitor compound) favorable/permissive for kinase activity. In preferrred embodiments the sample is incubated for a time that allows for at least half of the substrate to be modified, assuming maximal kinase activity. After allowing sufficient time for the Mst1 kinase to have modified the substrate, the substrate is recovered and contacted with an antibody that binds specifically to the phosphorylated substrate, but not the non-phosphorylated substrate. In one embodiment, the antibody specifically binds to the peptide CSAPAPKKGSKK (SEQ ID NO: 3). Quantifying the amount of S14P antibody that is bound to the substrate peptide is a direct correlation of the level activity of the kinase in the sample. Similarly, stimulants of Mst1 activity can be identified using the same assay and identifying compounds that enhance the rate that the substrate gets phosphorylated.

In accordance with one embodiment of the present invention a method is provided for screening a library of compounds to isolate modifiers of Mst1 activity. The method comprises the steps of first providing an Mst1 substrate that has been equally portioned into separate reaction chambers (typically separate wells of a microtiter dish). Preferably, the Mst1 substrate is a protein/peptide comprising the sequence SAPAPKKGSKK (SEQ ID NO: 2) and in one embodiment the peptide is covalently bound to the surface of the microtiter dish either directly or indirectly through a linker. The Mst1 substrate is then contacted with the Mst1 enzyme, and members of the compound library to form a kinase reaction mixture. In one embodiment a single candidate modulating compound is add to each well, however in an alternative embodiment 2 or more compound library members are added to each individual reaction chamber. The kinase reaction mixture comprising the Mst1 substrate, Mst1 and the candidate modulating compound is incubated under conditions typically permissive for Mst1 kinase activity (i.e. in the absence of a Mst1 inhibitor). After an appropriate length of time sufficient to allow for phosphorylation of the Mst1 substrate the reaction is stopped and the substrate is recovered. The extent that the substrate is phosphorylated (relative to an established standard curve for Mst1 activity or a control reaction run simultaneously) is directly related to the modulating effect of the added library compound on the activity of the Mst1 enzyme. Such Mst1 modulating compounds can be used as therapeutics to treat diseases associated with inappropriate apoptotic activity, including cancer.

Measuring the phosphorylation of the Mst1 substrate can be accomplished using a polypeptide comprising the amino acid sequence SAPAPKKGSKK (SEQ ID NO: 2) as the substrate and the S14P antibody. In accordance with one embodiment the substrate is linked to the surface of the reaction vessel directly or indirectly (i.e. through a linking moiety) via covalent, ionic, hydrogen or other chemical bond. In one embodiment the substrate is bound to the surface of the reaction chamber by adding the Mst1 substrate to the reaction chamber and incubating in PBS overnight at 4° C. Alternatively, the peptides are covalently linked to the surface of the reaction chamber using standard reactive groups and reactions known to those skilled in the art. After incubating the substrate in the presence of the Mst1 enzyme and the potential inhibitor, the reaction mixture is removed from the reaction chamber and the chamber is optionally washed with buffer. The S14P antibody is then added to the reaction chambers under conditions that allow for specific binding to its substrate and then the reaction chambers are washed with buffer to remove non-specifically bound material. In accordance with one embodiment specific binding of the antibody to the peptides is detected through an ELISA reaction. In an alternative embodiment the substrate is not bound to the reaction chamber, and the specific binding of the antibody to the peptides is detected by immunoprecipatation.

The antibodies of the present invention have a number of potential uses including their use as markers of apoptosis. Apoptosis has known to occur in cancer, neurodegenerative diseases, stoke etc. In addition the marker can also be used to assess and monitor the progress of a therapeutic treatment. The S14P antibody can also be used as a marker of the changes in chromatin that occurs during apoptosis. The antibodies can also be used to study the signals that are responsible for the histone modification (H2B ser 14 phosphorylation) and how this modification is involved in apoptosis.

In one embodiment of the present invention a kit is provided for detecting apoptotic cells. The kit comprises an antibody that specifically binds to a phosphorylated serine modified peptide comprising the sequence SAPAPKKGSKK (SEQ ID NO: 1). In one embodiment the kit further comprises an antibody that specifically binds to the non-phosphorylated amino terminus of the histone H2B peptide comprising the sequence SAPAPKKGSKK (SEQ ID NO: 2). In one embodiment the antibodies are attached to an insoluble support, wherein the support is either a monolithic solid or is in particular form. In one preferred embodiment the antibodies are monoclonal antibodies and in a further embodiment the antibodies are labeled. To this end, the antibodies of the present invention can be packaged in a variety of containers, e.g., vials, tubes, microtiter well plates, bottles, and the like. Other reagents can be included in separate containers and provided with the kit; e.g., positive control samples, negative control samples, buffers, cell culture media, etc.

In accordance with one embodiment the kits further comprise peptides that serve as substrates for kinase activity. More particularly, the peptides comprise the sequence SAPAPKKGSKK (SEQ ID NO: 2). In one preferred embodiment the peptide substrate is covalently bound either directly or through a linking moiety to a solid substrate.

The kits of the present invention may further comprise reagents for detecting the antibody once it is bound to the target antigen. Optionally, reagents (pepsin, dilute hydrochloric acid) for treating cells or tissue to render nuclear proteins accessible for immunological binding may also be included, as may immunofluorescent detection reagents (an anti-immunoglobulin antibody, or anti-histone H2B antibody, derivatized with fluorescein or rhodamine, or a biotinylated anti-immunoglobulin antibody together with avidin or streptavidin derivatized with fluorescein or rhodamine), immunohistochemical or immunocytochemical detection reagents (an anti-immunoglobulin antibody, or anti-histone H2B antibody, derivatized with alkaline phosphatase or horseradish peroxidase, or a biotinylated anti-immunoglobulin antibody together with avidin or streptavidin derivatized with alkaline phosphatase or horseradish peroxidase). In one embodiment, the kit includes one or more reagents for immunoperoxidase staining (an anti-immunoglobulin antibody derivatized with horseradish peroxidase, or a biotinylated anti-immunoglobulin antibody together with avidin or streptavidin derivatized with horseradish peroxidase), together with a chromogenic substrate therefor (e.g., diaminobenzidine).

### EXAMPLE 1

20 Identification of Phosphorylated Serine 14 of H2B as a Marker for Apoptosis

Experimental Procedures

Cell culture, drug and UV treatment, and harvesting for protein and DNA: HL-60 cells were cultured in RPMI with 10% FBS whereas 293T, HeLa, HepG2, and IMR90 were grown in DMEM with 10% FBS. Etoposide, anisomycin, 25′ and anti-Fas were purchased from Sigma. To induce apoptosis, drugs were used in these concentration 20 ug/mL for etoposide, 25 ng/mL anisomycin and 15 ng/mL anti-Fas. For UV induction of apoptosis, 40-100 J/m² was used (Biorad GS Gene Linker UV chamber). After induction of apoptosis the growth media was changed and harvested at various times afterward. Control cells were either treated with DMSO or mock-treated.

After the desired treatment, cells were subjected to any of the following: resuspension in SDS-lysis buffer (Laemmli's sample buffer), nuclear extraction, acid extraction for histones, or genomic DNA extraction. Nuclei were isolated by lysis in detergent and low speed spin as described in Strahl et al., (2001) Curr Biol 11, 996-1000. These nuclei can either be salt extracted for nuclear proteins (see below) or acid extracted in 0.4 N H₂SO₄. After incubating on ice for 2-4 hours, high speed centrifugation is done to remove the insoluble pellet. 5.4% of Percholic acid was added to the supernatant and the precipitated histones were resuspended in water.

Genomic DNA was harvested by lysing the cell pellet in 10 mM Tris pH 9.0, 1 mM EDTA, 10 mM NaCl, 1% w/v SDS, 1 mg/mL of proteinase K at 50° C. for 4-5 hours. To remove proteins, a phenol/chloroform extraction was performed and then the DNA was precipitated in 0.3 M sodium acetate and 70% Ethanol. After centrifugation, the pellet was resuspended in TE and the RNA was digested by adding 1 mg/mL Rnase A for 1 hour at room temp. DNA was separated in 1.2% agarose TAE gel.

Salt Nuclear Extraction and In-Gel Kinase Assay

Nuclei from HL-60 were extracted in 20 mM Hepes pH 7.8, 0.42 M NaCl, 1.5 mM MgCl₂, 0.2 mM EDTA, 0.5 mM PMSF, 0.5 mM DTT, 25% (v/v) glycerol for 3-4 hours on ice. After centifugation at 14K for 20 minutes, soluble fraction was isolated and used for in vitro kinase assay, in-gel kinase assay, and Western blotting (see below). SDS-PAGE was done as described by Laemmli. For in-gel assay, 10-12% SDS-PAGE gel was polymerized with 0.1 mg/mL chicken core histones or 0.1 mg/mL purified chicken H2B. In-gel was done as essentially described in (Sassone-Corsi et al., (1999) Science 285, 886-891. Briefly, nuclear extracts or fractions were run into these gels. Then to rid the SDS, the gel was washed repeatedly (three times) in 30 mM Tris HCl pH 7.4, 1 mM DTT, 0.1 mM EDTA, 20% (v/v) isopropanol for 20 minutes each time. To denature the proteins, the gel was incubated in 8 M urea, 30 mM Tris HCl pH 7.4, 1 mM DTT, 0.1 mM EDTA for one hour. The gel was then immersed in 30 mM Tris HCl pH 7.4, 5 mM MgCl₂, 2 mM MnCl₂, 1 mM DTT, 100 mM NaCl, 0.05% Tween 40 at 4° C. overnight to renature the proteins. After renaturation, an in vitro kinase reaction was performed using 50 mCi of g-ATP in 30 mM Tris HCl pH 7.4, 5 mM MgCl₂, 2 mM MnCl_{2, 1} mM DTT at 30° C. for 2 hours. The gel is then stained with Coomassie Blue, destained, and dried down for autoradiography.

Nuclear Extract Fractionation, In Vitro kinase Assay and Western Blotting

Nuclear extracts were fractionated in a superose 6 PC 3.2/300 column (Amersham/Pharmacia) using the Pharmacia SMART system. The eluent consists of 200 mM NaCl, 50 mM NaPO₄ pH7.0, 5 mM MgCl₂, 20% v/v glycerol. The flow rate was 40 mL/min at 4° C. The apoptotic fraction containing the caspase cleaved Mst1 comes off at molecular weight of approximately 66 kDa whereas, the full length Mst1 comes off at around 150 kDa. 10 mL of fractions were used for in vitro kinase reaction and Western blotting. For the in vitro kinase assay, 0.5 mg of histone H2B was used in 30 mM Tris pH 7.5, 5 mM MgCl₂, 10 mM DTT, 0.1 mg/mL microcystin, 10 mM ATP plus 0.5 mCi of g-P32 ATP. The reaction was incubated for 20 minutes at 30° C. and spotted on P81 filter paper. Then filter papers were washed in 0.75% H₃PO₄ and a scintillation counter was used to measure P³² incorporation. For non-radioactive reaction (cold), no g-P³² ATP was used and the reactions were run in an SDS-PAGE gel for Western blot analysis.

Western blot analysis is performed as described in (Briggs et al., (2001) Genes Dev 15, 3286-3295) 1:1000 of α-phos (S14) H2B, 1:2500 of α-N-Mst1, 1:400 of α-myc (9E10 from Santa Cruz), 1:800 of α-PARP (UBI), 1:1000 of α-phos H2A.X (UBI), and 1:5000 of ax-Acetyl H4 primary antibodies were used. HRP-conjugated rabbit secondary (Amersham Pharmacia) was used at 1:5000. For chemiluminescence, the ECL plus kit (Amersham Pharmacia) was used for detection.

Immunofluorescence on Apoptotic Cell Culture and Xenopus Tails

Cells were fixed in 4% paraformaldehyde for 10 minutes and then washed in PBS. 0.2% Triton X-100 for 5 minutes to permeablize cells and then cells were blocked in 2% goat serum PBS. Cells were incubated for 1 hour with 1:500-1:1000 of α-phos (S14) H2B at 37° C. Following incubation, cells were washed three times with block. After washing, cells were incubated for 1 hour in 1:500-1:800 of Goat anti-rabbit Cy3 conjugated secondary antibody and washed three times in block. The coverslip containing cells was then mounted in Vectashield mounting media containing DAPI.

Degenerating tails were cut off from stage 64 froglets and fixed in Bouin fixative (Sigma) for 2 hr, washed overnight in 70% ethanol, dehydrated, cleared in Histoclear and embeded in paraplast. 10 mm sections were de-paraffinated, hydrated and blocked for 30 min in 0.1% BSA in PBS-0.05% Tween 20. Sections were incubated for 30 min with α-phos (S14) H2B (1:5000 dilution in 1% BSA in PBS-0.05% Tween 20), washed twice, 10 min each, in PBS-Tween. After washing, sections were incubated for 30 min with anti-rabbit fluorescein conjugated secondary antibody (1:200 dilution in 1% BSA in PBS-Tween) and washed twice, 10 min each in PBS-Tween. Sections were mounted in Antifade (Molecular probes) containing Hoechst (2 ug/ml).

Transient Transfection, Immunoprecipitation, and Kinase Assay

Transfection was done according to the lipofectamine plus kit (Gibco Invitrogen). After 24-48 hours of transfection, 293T cells were harvested in 40 mM Hepes pH 7.5, 1% Triton X-100, 0.05% NP-40, 150 mM NaCl, 50 mM NaF, 1 mM EDTA, 1 mM EGTA, 10% v/v glycerol, 1 mM phenylmethylsulfonyl fluoride, 0.5 mg/mL microcystin, 1 mg/mL aprotinin, 1 mg/mL pepstain, 1 mg/mL leupeptin. Supernatant was saved after 20 min of centrifugation at 14,000 rpm. For immunoprecipitation (IP), 1 mg of α-myc (9E10 from Santa Cruz) was incubated with supernatant for 2 hours at 4° C. and then precipitated with protein G-Sepharose (Amersham Pharmacia) for another 2 hours at 4° C. IPs were washed in TBST (20 mM Tris pH 7.5, 150 mM NaCl, 0.1% Tween 20) for 5-6 times. Then the beads were incubated in 1 mg of H2B, 40 mM Hepes 7.5, 20 mM MgCl₂, 0.1 mg/mL microcystin, 10 mM ATP, 1 mCi g-P32 ATP for 30 minutes at 30° C. The reaction was separated on 15% SDS-PAGE gel and dried for autoradiography.

Results

Phosphorylation of H2B at Serine 14 is Strongly Induced in HL-60 Cells Undergoing Apoptosis

Previously, in vivo labeling studies show that H2B phosphorylation occurs specifically at the amino-terminus tail during apoptosis. Hence, a site-specific, H2B serine 14 phospho-specific antibody (S14P antibody) was generated to examine these relationships further. Initially, HL-60 (human leukemia cell line) cells were treated with etoposide (VP-16) to induce apoptosis. After treatment for various length of time, histones were isolated by acid extraction and examined by Western blot using the S14P antibody. H2B phosphorylation signal is strongly enhanced at around 4 hours post VP-16 treatment. To confirm the induction of apoptosis, soluble DNA was extracted from parallel samples at identical time points. The appearance of a ‘DNA ladder’, indicative of apoptotic DNA fragmentation at 4 hour post treatment, coincides precisely with the onset of H2B Ser14 phosphorylation. Furthermore, an increase of H2B S14 phosphorylation was observed that correlates with the exposure time to the etoposide. These data demonstrate that the two events are temporally linked.

To confirm that phosphorylation of H2B at serine 14 occurs specifically in apoptotic cells, VP-16 treated, HL-60 cells were immuno-stained with the S14P antibody. Strikingly, the majority of the S14P antibody signal is found localized to characteristic apoptotic chromatin bodies identified by DAPI staining. These data suggest that phosphorylation of H2B at serine 14 occurs specifically in apoptotic HL-60 cells. In contrast, other histone antibodies, including ‘mitotic’ H3 (Ser10) phospho antibodies, do not stain these apoptotic nuclei. As well, the S14P antibody fail to stain condensed mitotic chromosomes, suggestive of biological specificity (mitotic vs. apoptotic) of these distinct histone phosphorylation events. To rule out any unexpected effects of VP16, other apoptotic inducers were tested in different cell lines. As summarized in Table 1, different cell lines and well-known apoptotic inducers also led to H2B (Ser14) phosphorylation during apoptosis, albeit to variable extents. Hence, our data clearly demonstrate that histone H2B phosphorylation at serine 14 is associated with condensed apoptotic chromatin in multiple mammalian cell lines.

TABLE 1
Different Cell Lines Undergoing Apoptosis Display
Increase in H2B S14 Phosphorylation

| Cell Lines | | Levels of H2B S14 Phons |
|---|---|---|
| Tested | Cytotoxic Agents | Induction |
| HL-60 | Etoposide (VP16) | High |
| | UV | High |
| | Anti-Fas | High |
| | Anisomycin | High |
| NIH3T3 | UV | High |
| HeLa | UV | Medium |
| IMR90 | UV | Medium |
| HepG2 | MMS | Medium |
| 293T | UV | Low |

After Cytotoxic agent treatments, cells were harvested. The level of H2B S14 phosphorylation is determined by Western blots using ePhos (S14) H2B.

### H2B (Ser14) Phosphorylation is Increased During Program Cell Death During Developmental Sculpturing Events

To determine whether H2B (Ser14) phosphorylation is also associated with apoptosis in a more physiological model, this modification was examined in an animal where programmed cell death is well known and well characterized. Inspection of the primary amino acid sequence of histone H2B amino termini reveals that serine 14 is conserved among vertebrate species, including Xenopus. During metamorphosis, resorption occurs in Xenopus laevis tails and clusters of these cells die by developmentally programmed apoptosis. Degenerating tails from stage 64 froglets were examined by immunofluorescence using the S14P antibody. Signal from this antibody typically stains pockets of apoptotic cells in the tail and co-localizes precisely with the Hoechst-dense apoptotic condensed nuclei. These data indicate that H2B (Ser14) phosphorylation also occurs in apoptotic cells during normal Xenopus development. In contrast, no apoptotic staining is observed with the S14P antibody in Caenorhabditis or Drosophila consistent with the observation that this serine does not exist in these invertebrates.

Nuclear Extracts From Apoptotic Cells Contain a 34 kDa H2B Ser14 Kinase

To further understand the link between H2B (Ser14) phosphorylation and apoptosis, attempts were made to identify the kinase(s) responsible for this phosphorylation event. HL-60 cells were chosen as a starting point for these studies, since Ser14 phosphorylation of H2B is strongly increased in VP-16-treated HL-60 cells. Nuclear extracts from mock-and VP-16-treated HL-60 cells were used for preliminary kinase assays and Western blots. Firstly it was determined that ‘apoptotic’ nuclear extracts contain a H2B Ser14 kinase activity that is greatly increased in stimulated cells.

A histone H2B-based, in-gel kinase assay was used to search for, and ideally, determine the molecular weight(s) of potential apoptotic H2B kinases in crude apoptotic extracts. Using either H2B or mixtures of core histones as substrate, a prominent apoptotic-induced histone kinase was detected. While numerous bands appear in common between the H2B-containing and the ‘no substrate’ gels (likely due to autophosphorylation), one band is consistently detected in the H2B-containing gel that has an apparent molecular weight of approximately 34 kDa. These data suggest that stimulated (apoptotic) HL-60 nuclear extracts contain an induced, 34 kDa H2B kinase (hereafter 34H2BapoK).

Mst1 (Mammalian Sterile Twenty) is a caspase-activated Ser/Thr protein kinase of approximately 34 kDa whose activity plays causal role in inducing apoptosis under some biological settings. However, the physiological targets of this kinase, relative to programmed cell death, was previously unknown. To determine if 34H2BapoK is in fact Mst1, nuclear extracts prepared from VP-16-stimulated HL-60 cells were first probed with an amino terminal antibody to Mst1 (α-N-Mst1; this antibody detects the N-terminal catalytic domain of Mst1, (Graves et al., (1998) EMBO 17, 2224-2234)) to determine if the caspase-cleaved form of Mst1 is present in fractions containing 34H2BapoK as detected by the in-gel H2B kinase assay. Western blot analyses show that the cleaved form of Mst1 is present in these nuclear extracts, and its molecular weight is similar, if not identical, to the apoptotic-induced H2B kinase.

The Caspase-Cleavage Form of Mst1 Co-Fractionates With the H2B (Ser14) Kinase Activity

To further characterize the apoptotic-induced H2B kinase, the apoptotic nuclear extract was fractionated using a size exclusion column (Superose 6) to partially purify the kinase. Comparison of H2B kinase activities from ‘apoptotic’ and ‘normal’ nuclear extracts, showed that fraction 18 of the apoptotic extract has 2-3 fold enrichment in H2B kinase activity over the same fraction from mock-treated extracts. This enrichment is not due to increased autophosphorylation of polypeptides in fraction 18 as kinase assays performed without substrate did not exhibit changes between control and apoptotic fractions. While other fractions exhibit histone H2B kinase activity, no signiticant differences were detected between mock-treated and VP16-treated extracts suggesting that the apoptotic-induced H2B kinase is not present in these fractions. To confirm that the H2B kinase activity contained fraction 18 phosphorylates H2B at Ser14, kinase reactions using non-radioactive ATP and H2B as the substrate were performed and analyzed by western blot using the S14P antibody. As expected, only fraction 18 has the enhanced H2B serine 14 kinase activity.

Western blotting of the fractions immediately surrounding fraction 18 with the α-N-Mst1 show that only fraction 18 from apoptotic extracts contains the caspase-cleaved form of Mst1. Taken together, the coincidence of the increased H2B Ser14 kinase activity and the presence of the caspase-activated form of Mst1 in fraction 18 prepared from apoptotic nuclear extracts strongly suggests that Mst1-cleavage product is 34H2BapoK. To further substantiate this hypothesis, an in-gel kinase assay was perform with fraction 18 from normal and apoptotic nuclear extracts using chicken H2B as the substrate. The in-gel kinase assay shows that this fraction indeed contains a 34 kDa H2B kinase activity that is not present in the control fraction. Therefore, these data are consistent with the 34H2BapoK being the cleaved form of Mst1.

Mst1 can Phosphorylate Histone H2B at Serine 14 In Vitro and In Vivo

To directly test whether Mst1 can phosphorylate histone H2B, CMV driven plasmids containing either myc-tagged full length (FL), kinase dead (KD), or a C-terminal truncated form (DC) of Mst1 were transfected into 293T cells. Immunoprecipitation (IP) of these forms of Mst1, using antibodies against myc, were then analyzed by in vitro kinase assays performed in the presence of g(³²P)-ATP or with cold ATP and using histone H2B as substrate. Cold reactions were assayed by Western blotting with α-myc and α-phos (S 14) H2B. Strong phosphorylation of H2B was detected with both full length and DC Mst1, a result consistent with findings that DC Mst1 mimics the caspase-cleaved form of Mst1. In contrast, the kinase dead form Mst1 (which contains a single point mutation) did not phosphorylate H2B, arguing against the possibility of other histone kinases associating with the Mst1 IPs. To determine histone preferences by Mst1, similar IP kinase reactions were performed using individual core histone as substrates. Again, only Mst1 FL and DC show strong preference for histone H2B. Taken together, these data suggest that H2B is a physiologically-relevant nuclear substrate for Mst1 in vivo.

To test whether Mst1 can phosphorylate H2B at Ser14, similar IP kinase reactions were performed with H2B and non-radioactive ATP. Reactions were then analyzed by western blotting using α-myc for Mst1 protein expression level and the S14P antibody. As predicted, both Mst1 FL and DC phosphorylate H2B at Ser14 as detected by the S14P antibody whereas kinase dead Mst1 does not. Hence, full length and truncated forms of Mst1 can directly phosphorylate histone H2B at Ser14.

Mst1 can induce apoptotic-like chromatin condensation when overexpressed in HeLa cells. To determine if Mst1 can phosphorylate H2B when expressed in HeLa cells, full length and kinase dead versions of Mst1 were transfected into HeLa cells, and western blots of cell extracts were then probed with the S14P antibody. An increase in H2B Ser14 phosphorylation was only detected in cells harboring the Mst1 full length construct (WT). Considerably less signal is detected with the kinase dead mutant. These data suggest that Mst1 functions as an H2B Ser14 kinase in cells under these assay condition and that H2B might be an important physiological substrate for Mst1 as it relates to apoptotic chromosome condensation.

Kinetics of H2B Ser14 Phosphorylation is Similar to Cleavage of Mst1 During Apoptosis

If Mst1 cleavage is important in establishing H2B Ser14 phosphorylation during apoptosis, the kinetics of both events should be similar. HL-60 cells were induced to undergo apoptosis and were harvested at half-hour interval for western analysis. Time-course analyses (probing western blots with the S14P antibody and the α-N-Mst1 antibody) indicate that the onset of H2B phosphorylation coincides with the first appearance of the caspase-cleaved Mst1 at two hours post induction. As a marker for apoptosis, identical blots were also probed with A-PARP (poly (ADP-ribose) polymerase which gets cleaved as cells undergo apoptosis and isolated genomic DNA was evaluated for the appearance of characteristic DNA laddering. Interestingly, cleavage of PARP and the appearance of DNA ladder occur immediately after both H2B phosphorylation and cleavage of Mst1 (approximately 2.5 hr post induction). While timing differences may reflect differences in detection sensitivity between these apoptotic markers, the data suggest that H2B phosphorylation precedes DNA laddering and hence, may play a role in establishing apoptotic DNA fragmentation by mechanisms that remain unclear. More importantly, the onset of caspase-cleaved Mst1 is closely linked to the appearance of H2B Ser14 phosphorylation during the time course of apoptotic induction.

Comparison Between the Kinetics of H2B Serine 14 Phosphorylation and Other Histone Modifications During Apoptosis

Acetylation of histone H4 is decreased during apoptosis as cells undergo condensation and DNA fragmentation, and more specifically, histone H4 deacetylation occurs soon after the appearance of H2B phosphorylation. Thus, it seems unlikely that H2B phosphorylation results due to the loss of hyperacetylated (active) chromatin domains during apoptotic chromatin condensation. H2A.X phosphorylation has also been associated with double-strand DNA breaks. Since VP16 (etoposide) also causes DNA breaks, the kinetics of H2A.X phosphorylation were also evaluated. As expected, H2A.X phosphorylation occurs within one hour of VP16 treatment which suggests that DNA breaks, induced by VP16, occur early during the apoptotic pathway. Since H2B (Ser14) phosphorylation occurs later than H2A.X phosphorylation, but before (or concurrently with) other well known apoptotic markers, these data suggest that H2B phosphorylation is not simply associated with DNA breaks. Rather, H2B phosphorylation appears to be a unique chromatin marker for apoptosis in HL-60 cells.

H2B Ser14 Phosphorylation and Cleavage of Mst1 are Caspase 3 Dependent

Since caspases are involved in many integral parts of the apoptotic programs, we sought to determine to what extent, if any, H2B Ser14 phosphorylation is controlled by activated, effector caspases. In addition, Mst1 cleavage is dependent on caspase 3 and the N-terminal cleavage form of Mst1, missing nuclear export signals (NES; cleaved off by activated Caspase 3), is then translocated into the nucleus. The truncated Mst1 then travels into the nucleus and induces chromatin condensation and apoptosis.

If H2B (Ser14) phosphorylation is dependent on the cleavage of Mst1, this histone modification should be sensitive to documented caspase 3 inhibitors such as the DEVD peptide. To test this prediction, HL-60 cells were pre-incubated with varying amounts of z-DEVD-fmk (Trevigen) before subsequent treatment with VP16 for 4 hours. Cell extracts were then harvested for western analysis. Western blots using Mst1 N-terminal and H2B S14 phos antibodies showed that incubation with 200 mM of z-DEVD-fmk inhibited both Mst1 cleavage and H2B Ser14 phosphorylation. At a lower dose, 20 mM of z-DEVD-fink, only a modest decrease in the proportion of truncated Mst1 over full length Mst1 and H2B phosphorylation is observed. These data demonstrate that H2B Ser14 phosphorylation, like cleavage of Mst1 itself during apoptosis, is dependent on caspase 3. Accordingly, H2B is likely phosphorylated in vivo at Ser14 by the caspase 3-generated form of Mst1. Moreover, the present data indicate that H2B is a previously unrecognized nuclear substrate for this activity, and hint at the possibility that phosphorylation of H2B at Ser14 is part of an apototic ‘histone code’, at least in vertebrates.

1. An antibody that binds specifically to an antigen selected from the group consisting of CSAPAPKKGSKK (SEQ ID NO: 3), SAPAPKKGSKK (SEQ ID NO: 1) and SAPAPKKGSKK (SEQ ID NO: 2).

2. The antibody of claim 1, wherein the antibody is a monoclonal antibody.

3. The antibody of claim 1, wherein the antibody specifically binds to the sequence SAPAPKKGSKK (SEQ ID NO: 1).

4. The antibody of claim 1, wherein the antibody specifically binds to the sequence SAPAPKKGSKK (SEQ ID NO: 2).

5. The antibody of claim 1, wherein the antibody is labeled.

6. The antibody of claim 5, wherein the antibody is labeled with a fluorescent marker.

7. A composition comprising the antibody of claim 1 and a diluent or pharmaceutically acceptable carrier.

8. A purified peptide consisting of an 11 to 20 amino acid sequence wherein said amino acid sequence comprises a sequence selected from the group consisting of SAPAPKKGSKK (SEQ ID NO: 1) and SAPAPKKGSKK (SEQ ID NO: 2).

9. The purified peptide of claim 8 wherein the peptide consists of the sequence CSAPAPKKGSKK (SEQ ID NO: 3).

10. A method for detecting the presence of apoptotic cells in a vertebrate species, said method comprising the steps of
contacting a biological sample with an S14P antibody, wherein said sample is isolated from said vertebrate species and comprises chromatin; and
identifying immunocomplexes formed between the chromatin and the S14P antibody as an indicator of the presence of apoptotic cells in the vertebrate species.

11. The method of claim 10 wherein the S14P antibody is labeled.

12. The method of claim 10 wherein the biological sample consists essentially of purified chromatin isolated from the cells of the vertebrate species.

13. The method of claim 10 wherein the immunocomplexes are identified by immunoprecipitation.

14. The method of claim 10 wherein the S14P antibody is immobilized on a solid support; and the step of identifying the immunocomplexes further comprises the steps of washing the bound antibody with a buffered solution and then contacting the bound antibody with a second labeled antibody that specifically binds to histone H2B.

15. A kit for identifying apoptotic cells, said kit comprising an antibody that specifically binds to a peptide selected from the group consisting of CSAPAPKKGSKK (SEQ ID NO: 3) and SAPAPKKGSKK (SEQ ID NO: 1).

16. The kit of claim 15 further comprising a second antibody that specifically binds to SAPAPKKGSKK (SEQ ID NO: 2).

17. The kit of claim 15 further comprising a polypeptide comprising the amino acid sequence SAPAPKKGSKK (SEQ ID NO: 2).

18-20. Canceled.


SEQUENCE LISTING (3 sequences)

SEQ ID NO: 1 - protein, 11 aa, Homo sapiens
  MOD_RES (9)..(9): Phosphorylation
SAPAPKKGSKK

SEQ ID NO: 2 - protein, 11 aa, Homo sapiens
SAPAPKKGSKK

SEQ ID NO: 3 - protein, 12 aa, Homo sapiens
  MOD_RES (10)..(10): Phosphorylation
CSAPAPKKGSKK